(19) United States
(12) Patent Application Publication
(10) Pub. No.: US 2025/0359850 A1
(43) Pub. Date: Nov. 27, 2025

(54) SUPPLEMENTED ULTRASOUND

(71) Applicant: KONINKLIJKE PHILIPS N.V., EINDHOVEN (NL)

(72) Inventors: Hyeon Woo Lee, Cambridge, MA (US); Muhammad Usman Ghani, Boston, MA (US); Jonathan Fincke, Belmont, MA (US); Balasundar Iyyavu Raju, North Andover, MA (US)

(21) Appl. No.: 18/874,163

(22) PCT Filed: Jun. 10, 2023
(86) PCT No.: PCT/EP2023/065560

§ 371 Date: Dec. 12, 2024

Related U.S. Application Data
(60) Provisional application No. 63/353,286, filed on Jun. 17, 2022.

(51) Int. Cl. A61B 8/00

(52) U.S. Cl. CPC A61B 8/463

(57) ABSTRACT

An ultrasound system (101) includes a memory (151) that stores instructions; a processor (152) that executes the instructions; and a display (180). In response to being executed by the processor (152), the instructions cause the ultrasound system (101) to: capture ultrasound imagery during an ultrasound examination; identify anatomical features captured in the ultrasound imagery during the ultrasound examination; and generate ultrasound images supplemented a list of one or more anatomical features captured in the ultrasound imagery during the ultrasound examination.

### BACKGROUND

Current ultrasound systems display image acquisition information and ultrasound images on monitors at points-of-care during medical examinations. Image acquisition information is limited to information specifying characteristics of the ultrasound systems when the ultrasound images are taken, and may be useful as feedback to help ensure that the ultrasound images are of high-quality. However, the information provided on the monitors at the points-of-care does not include patient data and/or other types of information. Patient data and other types of information may be useful for subsequent reviewers viewing the ultrasound images. The subsequent reviewers may include personnel involved in follow-up medical care, quality control, billing, insurance and more. Information from medical examinations for subsequent reviewers should be as comprehensive and accurate as possible, and collecting such comprehensive and accurate information after-the-fact can be tedious and require many steps. For example, the information may have to be gathered from many different sources. Delays as well as errors such as coding errors and duplicating errors may result due to the number of and complexity of steps required to collect comprehensive and accurate medical examination information after-the-fact. Additionally, some steps such as for filing billing reports require a human to process medical reports to find information matching the ultrasound images. Furthermore, it is often difficult to measure whether a medical examination is complete without an expert reviewing the entire medical examination after-the-fact. Typically, even the image acquisition information shown with the ultrasound images on the monitors at the points-of-care cannot be extracted in a report format, so even the image acquisition information is extracted by a human or by using sophisticated algorithms such as optical character recognition (OCR) algorithms.

### SUMMARY

According to an aspect of the present disclosure, an ultrasound system includes a memory that stores instructions, a processor that executes the instructions, and a display. In response to being executed by the processor, the instructions cause the ultrasound system to: capture ultrasound imagery during an ultrasound examination to identify anatomical features captured in the ultrasound imagery during the ultrasound examination; and generate ultrasound images supplemented with a list of one or more anatomical features captured in the ultrasound imagery during the ultrasound examination.

According to another aspect of the present disclosure, a method for supplementing ultrasound images includes capturing ultrasound imagery during an ultrasound examination; identifying, by a controller with a processor executing instructions from a memory, anatomical features captured in the ultrasound imagery during the ultrasound examination; and generating ultrasound images supplemented with a list of one or more anatomical features captured in the ultrasound imagery during the ultrasound examination.

According to another aspect of the present disclosure, a controller for an ultrasound system includes a memory that stores instructions; and a processor that executes the instructions. In response to being executed by the processor, the instructions cause the controller to: control an ultrasound probe to capture ultrasound imagery during an ultrasound examination; identify anatomical features captured in the ultrasound imagery during the ultrasound examination; and generate ultrasound images supplemented with a list of one or more anatomical features captured in the ultrasound imagery during the ultrasound examination.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for supplemented ultrasound, in accordance with a representative embodiment.

FIG. 2 illustrates a method for supplemented ultrasound, in accordance with a representative embodiment.

FIG. 3 illustrates a user interface for supplemented ultrasound, in accordance with a representative embodiment.

FIG. 4 illustrates another user interface for supplemented ultrasound, in accordance with a representative embodiment.

FIG. 5 illustrates another system for supplemented ultrasound, in accordance with a representative embodiment.

FIG. 6 illustrates a computer system, on which a method for supplemented ultrasound is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, supplemented ultrasound may provide supplementation information on a display of an ultrasound system, and may provide such information as a supplement to ultrasound images for subsequent uses. The supplemental information may include image acquisition information, and may also include subject-specific information, ultrasound examination-specific information, information from monitors such as patient monitors, facility-specific information, medical care provider-specific information, and more.

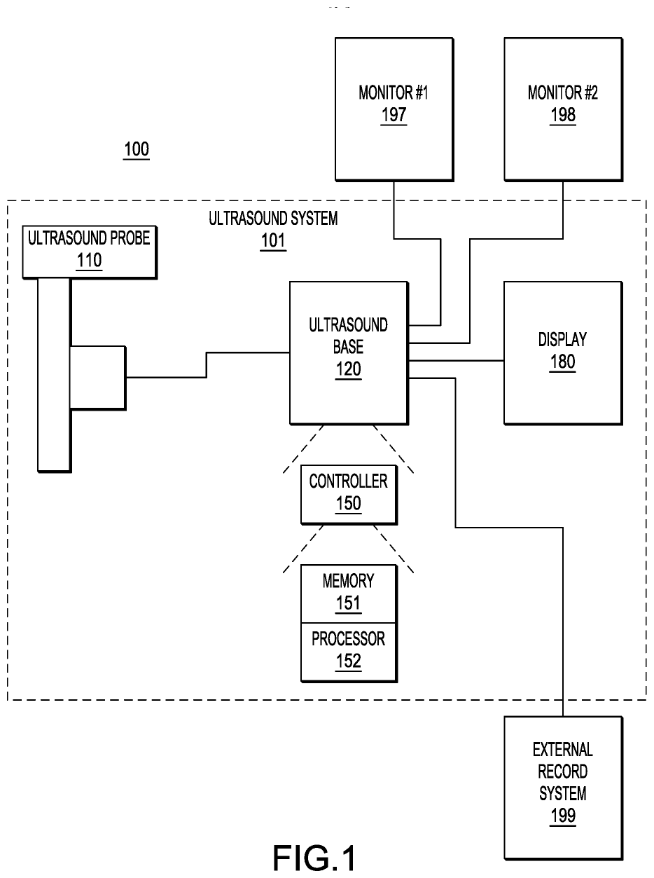

FIG. 1 illustrates a system 100 for supplemented ultrasound, in accordance with a representative embodiment.

The system 100 in FIG. 1 is a system for supplemented ultrasound and includes components that may be provided together or that may be distributed. The system 100 includes an ultrasound system 101 with an ultrasound probe 110, an ultrasound base 120 and a display 180. The system 100 also includes a monitor #1 197, a monitor #2 198, and an external record system 199.

The ultrasound probe 110 and the ultrasound base 120 may be provided as a cart-based ultrasound apparatus provided together at a subject bedside.

The ultrasound probe 110 is configured to transmit ultrasound imaging beams and receive and detect feedback from the transmitted ultrasound imaging beams. The ultrasound probe 110 may be a hand-operated probe or may be a body-mountable ultrasound transducer unit, e.g., a patch, for monitor purposes.

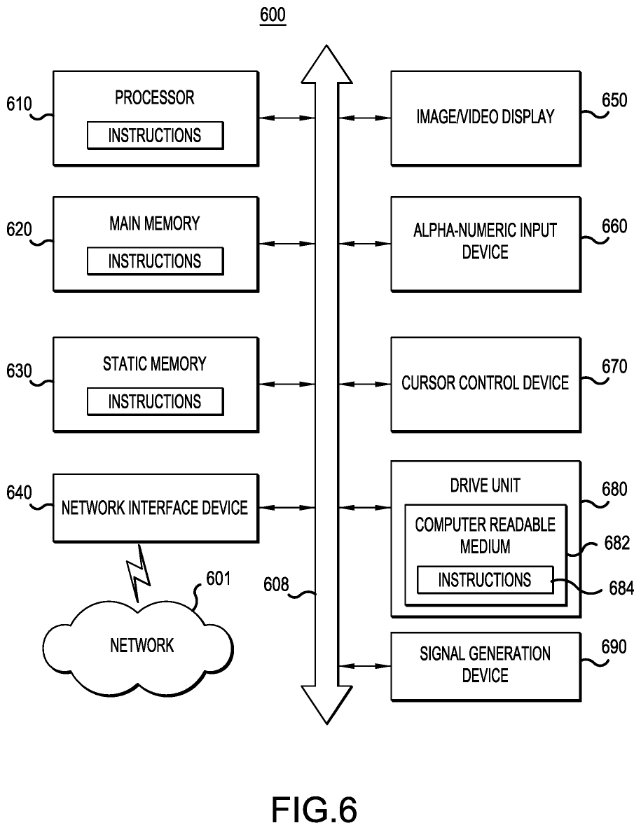

The ultrasound base 120 is configured for use to control ultrasound procedures and process feedback from ultrasound imaging beams transmitted from the ultrasound probe 110A. The ultrasound base 120 includes a controller 150, and the controller 150 includes at least a memory 151 that stores instructions and a processor 152 that executes the instructions. A computer that can be used to implement the ultrasound base 120 is depicted in FIG. 6, though an ultrasound base 120 may include more or fewer elements than depicted in FIG. 1 or FIG. 6. In some embodiments, multiple different elements of the system 100 in FIG. 1 may include a controller such as the controller 150.

The controller 150 and/or one or more other elements of the ultrasound base 120 may also include interfaces, such as a first interface, a second interface, a third interface, and a fourth interface. One or more of the interfaces may include ports, disk drives, wireless antennas, or other types of receiver circuitry that connect the controller 150 to other electronic elements. One or more of the interfaces may also include user interfaces such as buttons, keys, a mouse, a microphone, a speaker, a display separate from the display 180, or other elements that users can use to interact with the ultrasound base 120 such as to enter instructions and receive output.

The controller 150 may perform some of the operations described herein directly and may implement other operations described herein indirectly. For example, the controller 150 may indirectly control some operations such as by generating and transmitting content to be displayed on the display 180. The controller 150 may directly control other operations such as logical operations performed by the processor 152 executing instructions from the memory 151 based on input received from the ultrasound probe 110 and/or other electronic elements and/or users via the interfaces. Accordingly, the processes implemented by the controller 150 when the processor 152 executes instructions from the memory 151 may include steps not directly performed by the controller 150.

The display 180 is configured to display the ultrasound images supplemented with one or more types of information provided from and/or derived from the ultrasound probe 110, the ultrasound base 120, and/or sources external to the ultrasound system 101. The supplemented ultrasound images may be supplemented by integrating logical information with the ultrasound images, such as by auto-annotating information onto the ultrasound image, such as by superimposing. The display 180 may be local to the controller 150 or may be remotely connected to the controller 150. The display 180 may be connected to the controller 150 via a local wired interface such as an Ethernet cable or via a local wireless interface such as a Wi-Fi connection. The display 180 may be interfaced with other user input devices by which users can input instructions, including mouses, keyboards, thumbwheels and so on.

The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users.

The monitor #1 197 and the monitor #2 198 may monitor physiological characteristics of a subject undergoing an ultrasound examination. Examples of such monitored physiological characteristics may include glucose levels, blood pressure, heart rate, coagulation levels, electrocardiogramadings, oxygen saturation, temperature and more.

The external record system 199 is representative of record systems external to an ultrasound system 101. The external record system 199 may be or include an electronic medical record (EMR) system that stores subject information for a facility. The external record system 199 may also or alternatively be or include a picture archiving and communication system (PACS) in a medical facility such as a hospital. For example, the external record system 199 may provide information to the ultrasound base 120 on-demand, and the ultrasound base 120 may upload data to the external record system 199 in real-time or near-real time. Uploaded data may include supplemented ultrasound images described herein as image data, along with the logical data used to supplement the supplemental ultrasound images as logical data. Logical data may refer to data representing letters, numbers and symbols instead of pixel locations and pixel values.

Using the system 100, supplemental data that may be useful during a ultrasound examination may be displayed on the display 180 of the ultrasound system 101 at the point-of-care. After the ultrasound examination, supplemental ultrasound examination information may be readily extracted from the supplemental ultrasound images, such as when filing a billing report. Errors in reports such as billing and information reports may be avoided due to the integration of the supplemental information with the supplemented ultrasound images. The automated integration of the supplemental information may also reduce or entirely avoid some forms of unnecessary and tedious human labor otherwise required to gather information from different sources and formats. Because quality-control information may be integrated with the supplemental ultrasound images, quality control checks may be easily performed so as to detect when ultrasound examinations are incomplete or of low quality.

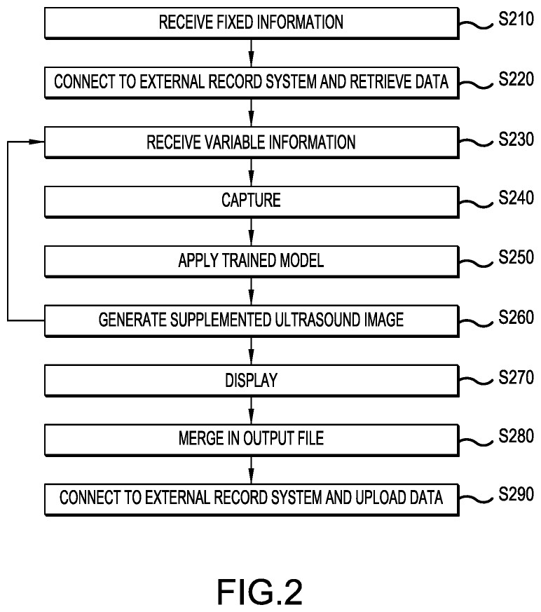

FIG. 2 illustrates a method for supplemented ultrasound, in accordance with a

representative embodiment. The steps illustrated and disclosed as part of FIG. 2 are provided as examples and in some cases, additional steps may be added. Likewise, in some examples, steps shown in FIG. 2 may not be part of a particular solution and inclusion here is provided as an example of one particular technique.

The method of FIG. 2 may be performed by the system 100 including the ultrasound base 120 with the controller 150.

The method of FIG. 2 starts at S210 by receiving fixed information. Fixed information may include subject data including name and/or subject identification number, subject age, and other fixed demographic information. Fixed information may also or alternatively include identification of a room and/or wing or department of a facility where the ultrasound examination is to be performed. Fixed information may be received by the ultrasound base 120 from the external record system 199 as in FIG. 1. Additionally or alternatively, subject identification information may be input to the ultrasound base 120 via a user interface of the controller 150 or of the display 180. Subject identification information and other fixed information may be received via a keypad or touchpad, or may be obtained through scanning a barcode or QR code such as on a temporary subject tag provided on a wrist of a subject upon admission to a facility. Scanning a barcode or QR code may be achieved through scanning an NFC tag. Also or alternatively, subject identification may be ported to the ultrasound base 120 from one or both of the monitor #1 197 and the monitor #2 198.

At S220, the method of FIG. 2 includes connecting to an external record system and retrieving data. For example, the ultrasound base 120 may connect to the external record system 199 and retrieve data for the subject, the ultrasound system 101, and/or the room in which the ultrasound examination is being performed.

At S230, variable information is received. The variable information received at S230 is separate from the data retrieved at S220. The variable information received at S230 may be imported from a monitor. For example, monitor #1 197 and/or monitor #2 198 may provide data to the ultrasound base 120 periodically, or dynamically when relevant data is generated at or received by the monitor #1 197 and/or monitor #2 198.

At S240, an ultrasound image is captured. The ultrasound image may be captured by the ultrasound probe 110 emitting ultrasound imaging beams and receiving and detecting feedback from the transmitted ultrasound imaging beams. The ultrasound probe 110 may provide data of the emitted ultrasound imaging beams and the received and detected feedback to the ultrasound base 120, and the ultrasound base may generate ultrasound images.

At S250, a trained model is applied to the ultrasound image captured at S240. The trained model may comprise an artificial intelligence (AI) model designed to detect corresponding anatomy of a subject. For example, the trained model may comprise a trained deep learning model. The following description will reference a trained deep learning model for the sake of consistency rather than as a specific requirement for a trained model described herein. The trained deep learning model may also be used to identify scanning views and determine when a scan of an imaging scanning view is complete. The trained deep learning model may be used to identify anatomical organs present in the ultrasound image captured at S240. The trained deep learning model may identify scanning views captured during the ultrasound examination and then identify anatomical features such as anatomical organs based on the identified scanning vies. The trained deep learning model may output information identifying the scanning view(s), completion status information for each scanning view, and a list of detected anatomy for each scanning view. Scanning views may also be referred to as zones, and correspond to predefined regions of anatomy. Some types of ultrasound systems use such scanning views to logically ensure completion of ultrasound imaging sessions. The imaging scanning views may be predetermined for a system such as for FAST (focused assessment with sonography in trauma) examinations, and the completion status information for each scanning view may help provide an overview of the quality of the ultrasound examination.

Trained models may be applied to captured ultrasound imagery. For example, in response to being executed by the processor 152, instructions may cause the ultrasound system to identify a scanning view captured during the ultrasound examination by applying trained models to the ultrasound images. The ultrasound images may be supplemented with the scanning view.

The trained deep learning model may detect anatomical organs and check whether a complete set of anatomical organs has been scanned and detected in order to confirm completion of a scan of a scanning view. The trained deep learning model may classify the relative completeness of the scanning view, anywhere from a binary complete/incomplete classification to a sliding scale such as a percentage complete. The scanning view classification may be used as a precursor to identifying which anatomical organs still need to be scanned in order to declare completion of a ultrasound examination. Afterwards, the trained deep learning model may detect anatomical organs that are necessary for each scanning view.

At S260, a supplemented ultrasound image is generated. The trained deep learning model may also be used to auto-annotate scanning view information onto the ultrasound image in order to create a supplemented ultrasound image. Auto-annotated information may include lists of detected anatomical organs detected by the trained deep learning model. The supplementing may be provided by auto-annotating information onto the ultrasound image, such as by superimposing. The supplemental information may include auto-annotated information such as lists of anatomical features present in ultrasound images, and scanning views and corresponding completion information indicating the relative completeness of scans for each scanning view. The supplemental information may also include information from the monitor #1 197 and/or from the monitor #2 198, as well as information from the external record system 199 in FIG. 1. The supplemented ultrasound image may be used for subsequent processes, including subsequent medical care, quality control checks, billing processing and insurance processing. The supplemental information may also be used to populate a template, and may also be provided separately from the supplemented ultrasound image as a logical data set that can be exported directly into systems such as billing and insurance systems. Templates may be provided for subject data and ultrasound examination information, and the templates may be customizable by users for different types of quality checks and/or billing systems and/or insurance systems.

The process from S230 to S260 may be repeated during the ultrasound examination, and may also proceed after each supplemented ultrasound image is generated at S260. For example, dozens or even hundreds of supplemented ultrasound images may be generated during a ultrasound examination performed using the ultrasound system 101.

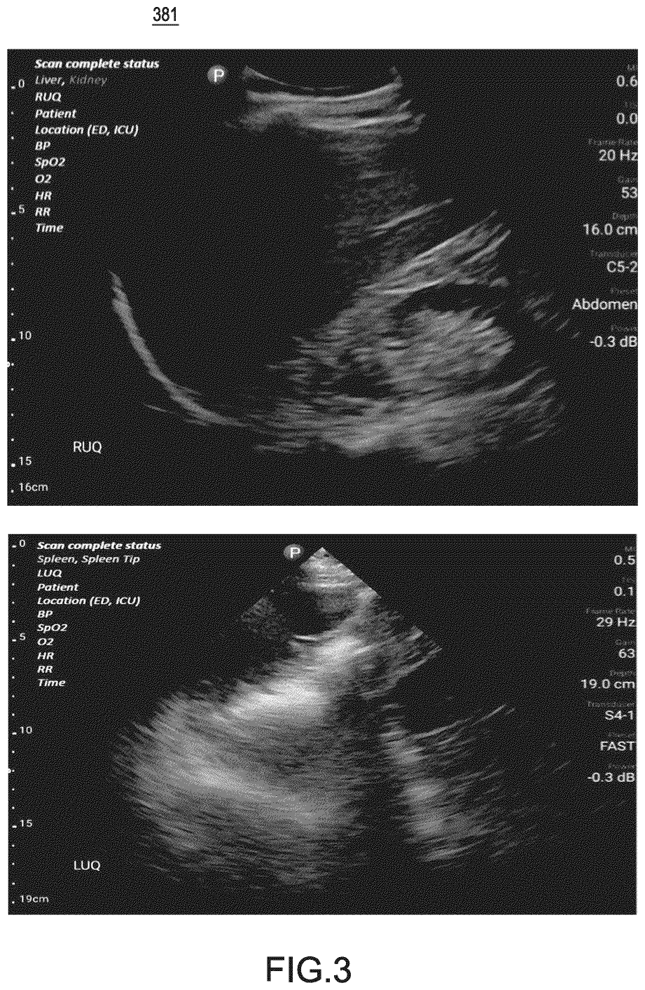

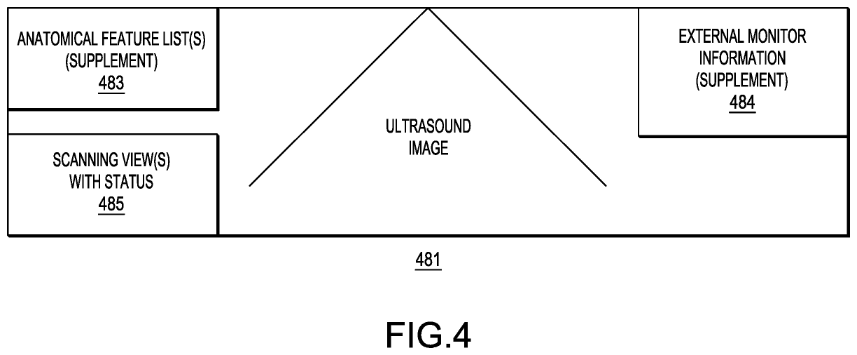

At S270, the supplemented ultrasound image is displayed. The supplemented ultrasound image(s) may be displayed on the display 180 of the ultrasound system 101. The display 180 may display information including indicators of completion of one or more scans for one or more scanning views. The supplemented ultrasound image(s) may also be stored, transmitted/transferred, and/or output as printed images via an image printer. Examples of supplemented ultrasound images are shown in and described with respect to each of FIG. 3 and FIG. 4 below.

At S280, the supplemented ultrasound image is merged in an output file, such as with the logical information used to supplement the original ultrasound image along with identifications of the source(s) of such supplemental information. The ultrasound system 101 may merge the ultrasound images in the output file with subject-specific information, with examination-specific information, and with information specific to medical personnel who administer the ultrasound examination. The merged ultrasound images may be raw ultrasound images or supplemented ultrasound images.

At S290, the method of FIG. 2 includes connecting to the external record system and uploading data. For example, the ultrasound base 120 may connect to the external record system 199 and upload some or all of the supplemented ultrasound images from an ultrasound examination, along with corresponding sets of logical information used to supplement the ultrasound images. S290 may be performed repeatedly so that supplemented ultrasound images and corresponding logical information are uploaded one at a time or in subsets of an overall group to be uploaded, or S290 may be performed at the end of the method in FIG. 2 so that all supplemented ultrasound images and corresponding logical information are uploaded one time as a batch.

FIG. 3 illustrates a user interface for supplemented ultrasound, in accordance with a representative embodiment.

In FIG. 3, two supplemented ultrasound images are shown stacked vertically on a user interface 381. However, two or more supplemented ultrasound images may also or alternatively be arranged horizontally in a row or in another arrangement on the user interface 381. The supplemental information provided on the user interface 381 may be customized by users, and may be readily printed, converted to a file format such as PDF and saved, and shared such as via upload to the external record system 199 in FIG. 1.

The user interface 381 in FIG. 3 is provided in an ultrasound system such as the ultrasound system 101 in FIG. 1. A list of information is integrated with (e.g., superimposed onto) the supplemented ultrasound image on/in the user interface 381. In FIG. 3 the ultrasound image and integrated information may be specific to a FAST ultrasound examination, though supplemented ultrasound as described herein is not limited to FAST ultrasound examinations or any particular form of ultrasound examinations.

The user interface 381 depicts how different information may be displayed as supplemental information on a display of an ultrasound system, such as the display 180 in FIG. 1. An ultrasound image may be provided with image acquisition information and also subject data, ultrasound examination information and more. Additionally, FAST examination scanning view information may be provided via the user interface 381 to provide a full picture of an ultrasound examination, including completion information for one or more scanning views.

Including subject data and automatically-acquired scanning view information in one location enables flawless communication between physicians, departments, and even hospitals such as level 1 to level 3 trauma centers. The quality of the ultrasound images and ultrasound examinations may be quickly explained using the supplemental information in/on the ultrasound images. The supplemental information may include automatically-acquired scanning view information and templated lists such as for anatomical information, along with metrics for completeness of FAST examinations that are required for quality checks. As a result, departments such as emergency rooms may readily hand-off subjects to departments such as ICU insofar as the receiving departments may readily observe subject history from the supplemented ultrasound images.

Additionally, the supplemented ultrasound images in FIG. 3 may include templated information that is usable for post-examination operations such as billing reports or quality analysis. Medical data from different sources may be coupled with ultrasound examination information in one system. The unified packaged data from separate sources may also enhance cloud data transfers, such as when subject information is moved to and from cloud storage.

FIG. 4 illustrates another user interface for supplemented ultrasound, in accordance with a representative embodiment.

In FIG. 4, a user interface 481 includes ultrasound images supplemented with identified scanning views and anatomical feature list(s) 483, external monitor information 484, and scanning view(s) with status 485. The anatomical feature list(s) 483 may include one or more lists of anatomical features specified for each of one or more scanning views listed in the scanning view(s) with status 485. The external monitor information 484 may include physiological information from monitors such as the monitor #1 197 and the monitor #2 198. Additional information which is not shown in FIG. 4 may also be used as supplemental information to supplement the ultrasound image shown on the user interface 481. As noted, the supplemented ultrasound image may be printed, saved and transferred with the supplemental information integrated with the ultrasound image as well as provided separately as logical information provided separate from image data of the supplemented ultrasound image. Additionally, the anatomical feature list may dynamically update as the ultrasound imagery changes either as a result of the subject moving or the probe being moved to capture a different zone (scanning view) of the subject. As an example, a list initially displaying a particular anatomical feature may be updated during an ultrasound examination to provide a live representation and dynamically updating list of the anatomical features within a field of view and detected within the ultrasound imagery. In an example, the updated list may be provided for display so that a user may see a continuously updating list as the ultrasound examination is performed and anatomical features previously undetected are captured and/or identified. Likewise, an identified view associated with particular ultrasound imagery may be updated as the ultrasound examination is occurring such that a shift of view either due to the subject or the probe will result in an update to the view identified and potentially displayed. As an example, in response to being executed by the processor 152, instructions may cause the ultrasound system 101 to update the list of one or more anatomical features captured in the ultrasound imagery in response to the captured ultrasound imagery changing during the ultrasound examination thereby capturing at least one previously uncaptured anatomical feature.

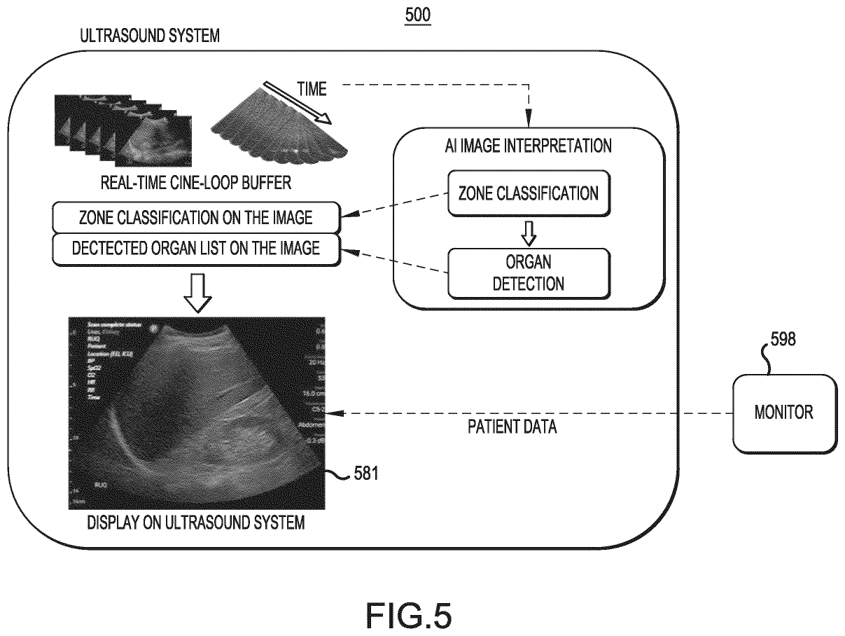

FIG. 5 illustrates another system for supplemented ultrasound, in accordance with a representative embodiment.

The ultrasound system 500 includes a user interface 581 and an external monitor 598. The user interface 581 of an ultrasound system 500 in FIG. 5 illustrates a supplemented ultrasound image with information provided from and/or derived from a pipeline of imported subject data.

Also in FIG. 5, a time-series of original ultrasound images may be fed to AI (artificial intelligence) image interpretation. The AI image interpretation may be implemented by a trained deep learning model executed as a program by a processor such as the processor 152 in FIG. 1. The AI image interpretation may include zone classification, and then organ detection. The zone classification may result in zone classification information provided as a supplement to the original ultrasound images. Zones may refer to imaging views with predetermined characteristics that are detectable by the AI image interpretation. Predetermined characteristics may include location, shape, bone/tissue delineation and more.

Examination-specific information such as scanning view information may be auto-annotated to the ultrasound image on the user interface 581. Scanning view information from the AI image interpretation may be provided so that ultrasound scanning personnel and physicians can subsequently readily estimate the overall quality of the FAST ultrasound examination. The organ detection from the AI image interpretation may result in a list of detected organs may be provided on a per-zone (per-imaging view) basis.

Also in FIG. 5, subject data from an external monitor 598 may be provided to supplement the original ultrasound images. The subject data may be variable data imported from one or more monitors such as the monitor #1 197 and/or the monitor #2 198 in FIG. 1 and/or fixed data imported from the external record system 199 in FIG. 1.

After completing an ultrasound examination, a user may customize the entry of subject data from one or more monitor and scanning view information from the trained deep learning model to populate or even create a template that is used for subsequently usage such as filling billing documents or insurance documents, or for processing quality checks. The template may be exported from the ultrasound system 500 to the external record system 199 as logical data in a user-defined document format along with the supplemented ultrasound image. That is, outputs may include data sets in image formats and other formats so that users can upload supplemented ultrasound images and corresponding data sets of logical information to data storage solutions and so that users can send supplemented ultrasound images and corresponding data sets to other physicians/hospitals.

In FIG. 5, a trained deep learning model may be applied to ultrasound images to infer in real-time which anatomical features are shown in the ultrasound images. The trained deep learning model may output two or more image interpretation results including scanning view and a detected anatomical organ list corresponding to detected organs from a list of expected organs for each scanning view. The trained deep learning model may output data used to annotate the scanning view information by extracting and encoding the features of the ultrasound image acquired during the ultrasound examination. The trained deep learning model may comprise a classification and detection model which can be used in other tasks such as binary image classification and other type of medically distinctive feature detection such as detection of inserted needles. Insofar as FAST examinations for trauma patients vary by scanning view, and insofar as important anatomical organs may require scanning depending on context, the integration of the supplemental information with ultrasound images may improve efficiencies and patient care as patients are moved between providers and departments. In FIG. 5, a quadrant (e.g., left upper quadrant) may list anatomical organs which require full scanning, such as spleen, spleen tip, diaphragm, and kidney. Another quadrant (e.g., right upper quadrant) may list other anatomical organs such as the liver, liver tip, diaphragm, and kidney as key marks identified in the ultrasound image. Using the supplemental information as in FIG. 5, medical personnel may quickly ascertain relevant information directly from supplemented ultrasound images as well as from corresponding data sets of logical data provided with the supplemented ultrasound images.

FIG. 6 illustrates a computer system, on which a method for supplemented ultrasound is implemented, in accordance with another representative embodiment.

Referring to FIG. 6, the computer system 600 includes a set of software instructions that can be executed to cause the computer system 600 to perform any of the methods or computer-based functions disclosed herein. The computer system 600 may operate as a standalone device or may be connected, for example, using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 may be considered a representative example of a processor of a controller and executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array

(PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, in response to being executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, supplemented ultrasound provides enhanced inter care team communication and improved efficiencies for processes after an ultrasound examination. Detected ultrasound examination quality metrics may be automatically displayed on an ultrasound system display, so that resultant ultrasound images may be supplemented with this and other types of information. The improved efficiencies may be realized by follow-up medical providers, quality control providers, billing providers, insurance providers and more. The data used to supplement ultrasound images may be provided from multiple different and diverse sources, so the automated integration of such supplemental information may result in improved efficiencies and may lead to higher quality care for the patient. Additionally, the automated integration during ultrasound examinations may help avoid inefficiencies otherwise resulting from integrating varying data formats and limited inter-device connectivity. As a result, ultrasound findings may be viewed with some or all relevant subject informant on one screen, and such information may include, for example, blood pressure, pulse oximetry readings, FAST examination imagery, subject name, patient insurance information, and image quality information.

Although supplemented ultrasound has been described with reference to several exemplary embodiments, it is understood that the words that have been used are words of description and illustration, rather than words of limitation. Changes may be made within the purview of the appended claims, as presently stated and as amended, without departing from the scope and spirit of supplemented ultrasound in its aspects. Although supplemented ultrasound has been described with reference to particular means, materials and embodiments, supplemented ultrasound is not intended to be limited to the particulars disclosed; rather supplemented ultrasound extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An ultrasound system, comprising:
a memory that stores instructions;
a processor that executes the instructions; and
a display, wherein in response to being executed by the processor, the instructions cause the ultrasound system to:
capture ultrasound imagery during an ultrasound examination;
identify anatomical features captured in the ultrasound imagery during the ultrasound examination; and
generate ultrasound images supplemented with a list of one or more anatomical features captured in the ultrasound imagery during the ultrasound examination.

2. The ultrasound system of claim 1, wherein in response to being executed by the processor, the instructions cause the ultrasound system further to:
receive information from an external monitor;
wherein the ultrasound images are supplemented with the information received from the external monitor.

3. The ultrasound system of claim 2, wherein in response to being executed by the processor, the instructions cause the ultrasound system further to:
display the ultrasound images supplemented with the information received from the external monitor and the list of one or more anatomical features captured in the ultrasound imagery during the ultrasound examination.

4. The ultrasound system of claim 1, wherein in response to being executed by the processor, the instructions cause the ultrasound system further to:
identify a scanning view captured during the ultrasound examination by applying trained models to the ultrasound images; and
supplement the ultrasound images with the scanning view.

5. The ultrasound system of claim 1, wherein in response to being executed by the processor, the instructions cause the ultrasound system further to:
identify anatomical organs captured during the ultrasound examination; and
supplement the ultrasound images with the anatomical organs as one or more anatomical features captured in the ultrasound imagery during the ultrasound examination.

6. The ultrasound system of claim 1, wherein in response to being executed by the processor, the instructions cause the ultrasound system further to:
connect to an external record system during the ultrasound examination to retrieve data, and
supplement the ultrasound images with the data retrieved from the external record system.

7. The ultrasound system of claim 1, wherein in response to being executed by the processor, the instructions cause the ultrasound system further to:
connect to an external record system during the ultrasound examination to retrieve data, and
upload data to the external record system during the ultrasound examination.

8. The ultrasound system of claim 1, wherein in response to being executed by the processor, the instructions cause the ultrasound system further to:
update the list of one or more anatomical features captured in the ultrasound imagery in response to the captured ultrasound imagery changing during the ultrasound examination thereby capturing at least one previously uncaptured anatomical feature.

9. The ultrasound system of claim 1, wherein in response to being executed by the processor, the instructions cause the ultrasound system further to:
merge the ultrasound images in an output file with subject-specific information.

10. The ultrasound system of claim 1, wherein in response to being executed by the processor, the instructions cause the ultrasound system further to:
display information indicating completion of one or more scans for one or more scanning view captured during the ultrasound examination.

11. The ultrasound system of claim 1, wherein supplementing the ultrasound images comprises populating a template with the list of one or more anatomical features.

12. A method for supplementing ultrasound images, the method comprising:
capturing ultrasound imagery during an ultrasound examination;
identifying, by a controller with a processor executing instructions from a memory, anatomical features captured in the ultrasound imagery during the ultrasound examination; and
generating ultrasound images supplemented with a list of one or more anatomical features captured in the ultrasound imagery during the ultrasound examination.

13. The method of claim 12, further comprising:
receiving information from an external monitor; and
displaying the ultrasound images supplemented with the information received from the external monitor and the list of one or more anatomical features captured in the ultrasound imagery during the ultrasound examination.

14. The method of claim 12, further comprising:
identifying a scanning view captured during the ultrasound examination by applying trained models to the ultrasound images; and
supplementing the ultrasound images with the scanning view.

15. The method of claim 12, further comprising:
identifying anatomical organs captured during the ultrasound examination; and
supplementing the ultrasound images with the anatomical organs as one or more anatomical features captured in the ultrasound imagery during the ultrasound examination.

16. The method of claim 12, further comprising:
updating the list of one or more anatomical features captured in the ultrasound imagery in response to the captured ultrasound imagery changing during the ultrasound examination thereby capturing at least one previously uncaptured anatomical feature.

17. The method of claim 12, further comprising:
displaying information indicating completion of one or more scans for one or more scanning views captured during the ultrasound examination.

18. A controller for an ultrasound system, comprising:
a memory that stores instructions; and
a processor that executes the instructions, wherein in response to being executed by the processor, the instructions cause the controller to:
control an ultrasound probe to capture ultrasound imagery during an ultrasound examination;
identify anatomical features captured in the ultrasound imagery during the ultrasound examination; and
generate ultrasound images supplemented with a list of one or more anatomical features captured in the ultrasound imagery during the ultrasound examination.

19. The controller of claim 18, wherein in response to being executed by the processor, the instructions cause the controller further to:
identify a scanning view captured during the ultrasound examination by applying trained models to the ultrasound images; and
supplement the ultrasound images with the scanning view.

20. The ultrasound system of claim 18, wherein in response to being executed by the processor, the instructions cause the ultrasound system further to:
identify anatomical organs captured during the ultrasound examination; and
supplement the ultrasound images with the anatomical organs as one or more anatomical features captured in the ultrasound imagery during the ultrasound examination.
